(19) United States
(12) Patent Application Publication
(10) Pub. No.: US 2006/0229502 A1
(43) Pub. Date: Oct. 12, 2006

(54) Portable medical diagnostic apparatus

(75) Inventors: Neil Pollock, Royston (GB); Adrian J. Streeter, Peterborough (GB); Jamie G. Wehbeh, Suffolk (GB)

Correspondence Address: Toby H Kusmer;McDermott Will & Emery, 28 State Street, Boston, MA, 02109, US

(73) Assignee: BAYER HEALTHCARE LLC, Elkhart, IN (US)

(21) Appl. No.: 10/558,450

(22) PCT Filed: Jun. 3, 2004
(86) PCT No.: PCT/US04/17346

§ 371 Date: Nov. 28, 2005

Related U.S. Application Data
(60) Provisional application No. 60/475,352, filed on Jun. 3, 2003.

(51) Int. Cl. G01N 33/00; A61B 5/00; G06F 1/16

(52) U.S. Cl. 600/300; 361/686; 436/95

(57) ABSTRACT

A medical diagnostic apparatus including a housing, a sensor assembly located within the housing and including a temperature sensing element, and at least one thermal seal compressed between the sensor assembly and the housing and separating the temperature sensing element from heat-generating internal components of the apparatus. A rigid printed circuit board (PCB) and a rigid frame are also positioned within the housing and secured together with the housing to provide the apparatus with improved stiffness and torsional rigidity. A docking station for use with the apparatus defines a pocket having a convex projection that mates with a concave depression of the apparatus during docking, and the medical diagnostic apparatus includes at least one electrically conductive contact that contacts an electrically conductive contact of the docking station during docking.

### CROSS-REFERENCE to RELATED APPLICATIONS

The present application claims priority from co-pending provisional U.S. Patent Application Ser. No. 60/475,352, filed Jun. 3, 2003 (Attorney docket number BYRK-28PR), which is incorporated herein by reference in its entirety.

### FIELD of the DISCLOSURE

The present disclosure relates to a medical diagnostic apparatus and, more particularly, to a portable medical diagnostic apparatus. Even more particularly, the present disclosure relates to a portable glucose meter having improved rigidity, improved heat insulating properties, and an improved docking station.

### BACKGROUND of the DISCLOSURE

Blood glucose meters are medical diagnostic instruments used to measure the level of glucose in a patient's blood. Some meters include sensor assemblies that determine glucose levels by measuring the amount of electricity that can pass through a sample of blood, and other meters include sensor assemblies that measure how much light reflects from the sample. A computer processor of the meter then uses the measured light or electricity from the sensor assembly to compute the glucose level and displays the glucose level as a number.

Generally, to operate a blood glucose meter, a patient or caregiver, such as a nurse or doctor, deposits a drop of the patient's blood onto a disposable cartridge or pad. The disposable cartridge along with the drop of blood is then inserted into a slot or port located on the blood glucose meter, whereupon the sensor assembly of the blood glucose meter tests the blood located on the disposable cartridge in order to determine the level of glucose in the blood. Upon determining the level of glucose in the blood, the blood glucose meter displays this information along with other information on a screen located on the blood glucose meter. Many glucose meters also include switches for allowing a user to input information or queries into the meter. Preferably, glucose meters are small enough and light-weight enough to be portable and conveniently carried by a user.

Since it is important that a glucose meter is small and light-weight enough to be easily carried (e.g., about the size of a personal digital assistant or a cellular telephone), it is also important that the glucose meter is strong enough and rugged enough (e.g., “ruggedized”) to withstand being accidentally dropped and continue to function properly. For example, it is desirable for a portable glucose meter to withstand being accidentally dropped from a height of at least about five feet, and not be damaged and be able to continue to function properly.

It is also important that a glucose meter have good heat insulating properties to ensure accurate glucose measurements. The sensor assemblies of glucose meters often include one or more temperature sensing elements (e.g., a thermistor, thermometer, or thermocouple device) which monitor the ambient temperature to enable temperature correction of sensor signals. As with any chemical sensing method, transient changes in temperature during or between measurement cycles can alter background signal, reaction constants and/or diffusion coefficients. Accordingly, a temperature sensor is used to monitor changes in temperature over time. A maximum temperature change over time threshold value can then be used in a data screen to invalidate a measurement. Absolute temperature threshold criteria can also be employed, wherein detection of high and/or low temperature extremes can be used in a data screen to invalidate a measurement. The microprocessor of the glucose sensor can make a determination as to whether the temperature of the testing environment is within predetermined thresholds, and prohibit a user from running a test if accuracy would be negatively affected. It is important, therefore, that any temperature sensing elements of the glucose meter not be affected by heat generated within the glucose meter (e.g., by an liquid crystal display of the meter having heat-generating back lighting). The temperature sensing elements of the glucose meter should also have access to the ambient temperature surrounding the meter.

Preferably, a portable glucose meter is provided with a docking station (or cradle) for receiving the glucose meter and for providing electrical connections between the docking station and the glucose meter. The electrical connections can be used for recharging the portable glucose meter and for transferring data between the portable glucose meter and another device, such as a personal computer or modem. The docking station should easily receive the portable glucose meter and provide a reliable electrical connection. Like the portable glucose meter, the docking station should also be ruggedized and be able to withstand being accidentally dropped yet continue to function properly. For example, it is desirable for a docking station to withstand being accidentally dropped from a height of at least about five feet, and not be damaged and be able to continue to function properly. In addition, the electrical connectors of the docking station and the portable glucose meter should be able to withstand thousands (e.g., 9,000 to 18,000) of docking cycles and still provide a reliable electrical connection.

What is still desired, therefore, is a new and improved medical diagnostic apparatus, such as a glucose meter. Preferably, the new and improved glucose meter will be small enough and light-weight enough to be portable and conveniently carried by a user. In addition, the new and improved glucose meter will preferably be designed to withstand being accidentally dropped by a user and continue to function properly. Preferably, the new and improved glucose meter will also have good heat insulating properties to ensure accurate glucose measurements. The new and improved portable glucose meter will preferably include a docketing station that is itself ruggedized and provides an easy and reliable electrical docking connection with the glucose meter.

### SUMMARY of the DISCLOSURE

The present disclosure is directed to exemplary embodiments of a new and improved portable medical diagnostic apparatus, such as a glucose meter, and a docking station for use with the glucose meter.

One exemplary embodiment of the medical diagnostic apparatus includes a housing, a sensor assembly located within the housing and including at least one temperature sensing element mounted on an auxiliary printed circuit board (PCB), and at least one thermal seal compressed between the auxiliary PCB of the sensor assembly and the housing and separating the temperature sensing element from heat-generating internal components of the medical diagnostic apparatus. In addition, the auxiliary PCB of the sensor assembly is pressed against the housing to provide a substantially direct thermal coupling between the exterior of the medical diagnostic apparatus and the temperature sensing element.

Another exemplary embodiment of the medical diagnostic apparatus includes a housing, and a primary printed circuit board (PCB) positioned within the housing. The PCB is rigid, flat and has a length extending between opposing ends and a width extending between opposing sides. The apparatus also includes a liquid crystal display (LCD) positioned within the housing adjacent a window of the housing, wherein the LCD includes a length and a width that approximates the length and the width of the primary PCB, and a rigid frame supporting the LCD and having a length and a width that approximates the length and the width of the primary PCB. The rigid frame is secured to the primary PCB and at least one of the frame and the primary PCB are secured to the housing.

One exemplary embodiment of the docking station includes an external housing defining a pocket for receiving the medical diagnostic apparatus. The pocket includes a wall extending upwardly from a bottom end of the pocket for slidingly receiving the wall of the medical diagnostic apparatus when the apparatus is received in the pocket, and wherein the wall of the pocket includes at least one opening spaced from the bottom end of the pocket and a convex projection extending from the bottom end of the pocket to the opening in the wall of the pocket. The convex projection of the docking station is sized and shaped to mate with a concave depression of the apparatus when the apparatus is received in the pocket. The docking station also includes at least one electrically conductive contact extending through the opening of the housing of the docking station so that, when the apparatus is received in the pocket, the contact extending out of the docking station contacts a contact extending out of the medical diagnostic apparatus.

Among other aspects, benefits and advantages of the present disclosure, the new and improved glucose meter is small enough and light-weight enough to be portable and conveniently carried by a user. In addition, the new and improved glucose meter is designed to withstand being accidentally dropped by a user yet continue to function properly. The new and improved glucose meter also has good heat insulating properties to ensure accurate glucose measurements. Furthermore, the new and improved docketing station is itself ruggedized and provides an easy and reliable electrical docking connection with the glucose meter.

Additional aspects, benefits and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION of the DRAWINGS

Reference is made to the attached drawings, wherein elements having the same reference character designations represent like elements throughout, and wherein:

FIG. 1 is a perspective front and end view of an exemplary embodiment of a handheld glucose meter constructed in accordance with the present disclosure;

FIG. 2 is a perspective front and end view, in section, of the handheld glucose meter of FIG. 1;

FIG. 3 is an exploded perspective front and end view of the handheld glucose meter of FIG. 1, wherein an internal frame and a primary printed circuit board of the meter are shown;

FIG. 4 is a perspective front view of the internal frame and the primary printed circuit board of the handheld glucose meter of FIG. 1;

FIG. 5 is a perspective front and end view of the internal frame and the primary printed circuit board of the handheld glucose meter of FIG. 1;

FIG. 6 is an enlarged view of the portion of the handheld glucose meter contained in circle “FIG. 6” of FIG. 1, wherein thermal seals of the glucose meter are shown surrounding a sensor assembly of the meter;

FIG. 7 is a further enlarged view of the portion of the handheld glucose meter contained in circle “FIG. 6” of FIG. 1;

FIGS. 8 through 10 are perspective front and side views of the handheld glucose meter of FIG. 1 shown being received in a docking station constructed in accordance with the present disclosure;

FIG. 11 is a side elevation view, partially cut-away, of the handheld glucose meter of FIG. 1 shown received in the docking station of FIGS. 8 through 10; and

FIG. 12 is an enlarged view of the portion of the handheld glucose meter and the docking station contained in circle “FIG. 12” of FIG. 11.

### DETAILED DESCRIPTION of EXEMPLARY EMBODIMENTS

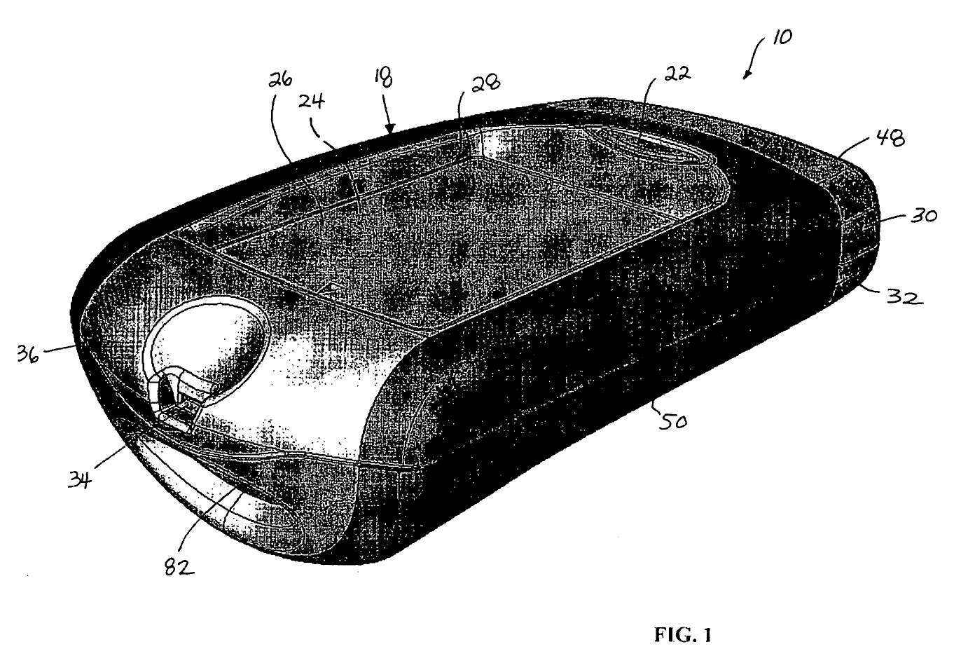

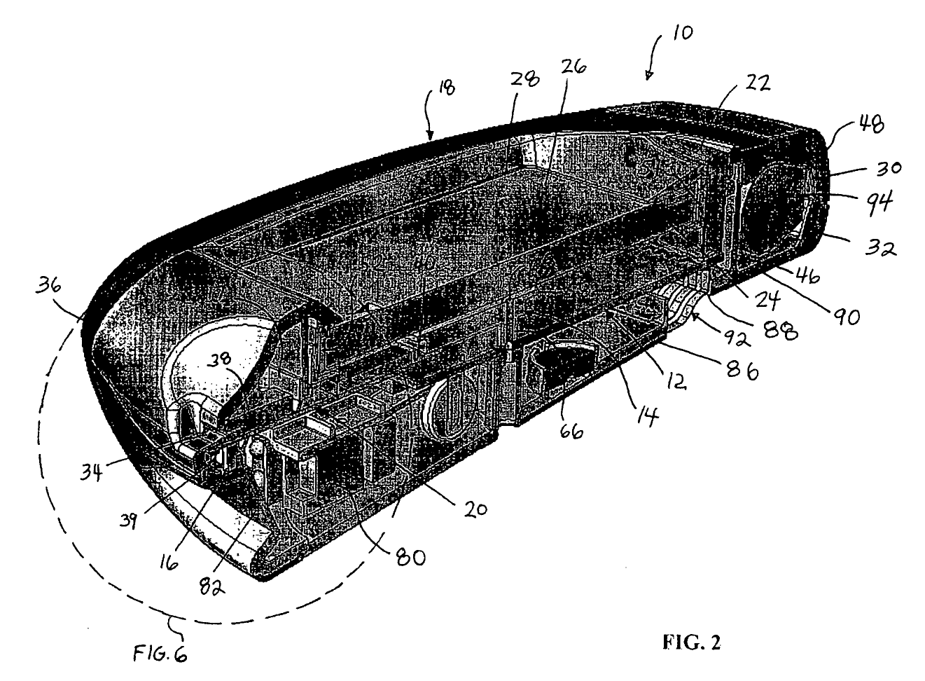

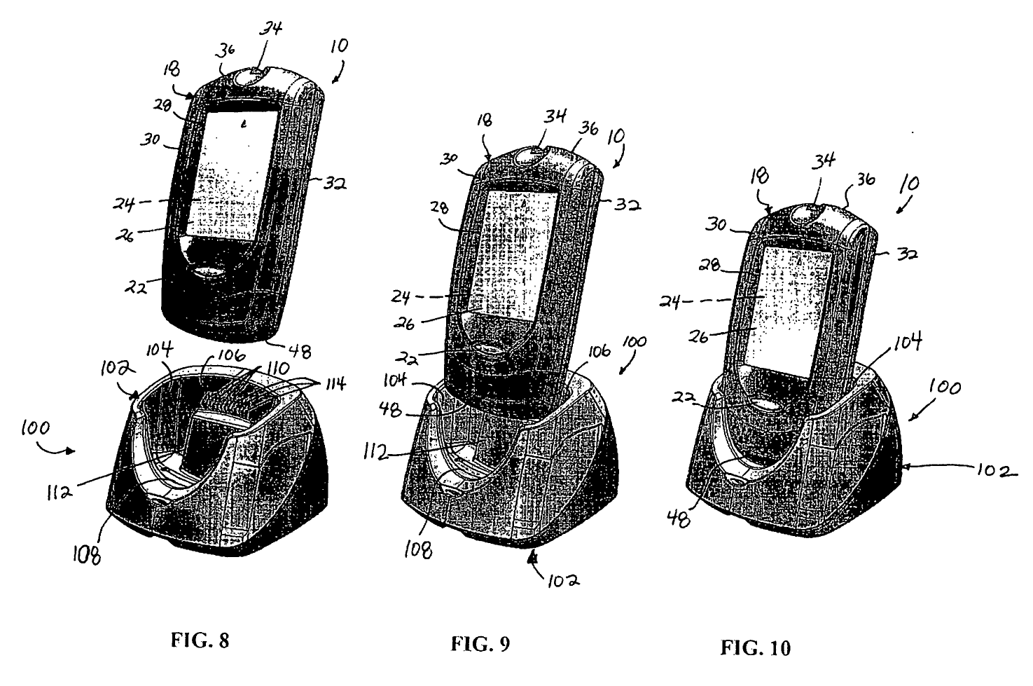

The present disclosure is directed to a new and improved portable medical diagnostic apparatus and a new and improved docking station (cradle) for use with the portable medical diagnostic apparatus. An exemplary embodiment 10 of a portable medical diagnostic apparatus, or parts thereof, constructed in accordance with the present disclosure is shown in FIGS. 1 through 12 of the attached drawings. Among other aspects, benefits and advantages of the present disclosure, the new and improved portable medical diagnostic apparatus 10 is designed to withstand being accidentally dropped by a user and continue to function properly. The medical diagnostic apparatus 10 can withstand being accidentally dropped because a primary printed circuit board (PCB) 12 of the apparatus is directly secured to an internal frame 14 of the apparatus, as shown best in FIGS. 2 through 5, to provide the apparatus with improved stiffness and torsional rigidity. The new and improved portable medical diagnostic apparatus 10 also has good heat insulating properties to ensure accurate operation. The heat insulating properties are provided by seals 16, 17 compressed between an external housing 18 and an internal sensor assembly 20 of the apparatus 10, as shown best in FIGS. 2, 3, 6 and 7. Furthermore, the new and improved docking station, an exemplary embodiment 100 of which is shown in FIGS. 8 through 12, is itself ruggedized and provides an easy and reliable electrical docking connection with the portable medical diagnostic apparatus 10.

Referring first to FIGS. 1 through 3, the exemplary embodiment of a portable medical diagnostic apparatus constructed in accordance with the present disclosure comprises a blood glucose meter 10. However, it should be understood that aspects of the present disclosure are applicable to portable medical diagnostic apparatuses other than blood glucose meters.

The glucose meter 10 generally includes the housing 18, which contains an on/off power switch 22, a display screen 24, and a user input device 26. In the exemplary embodiment shown, the display screen comprises a backlit liquid crystal display (LCD) 24 and the user input device comprises a touch screen 26 layered over the LCD. The housing 18 includes a window 28 for displaying and providing access to the LCD 24 and the touch screen 26.

The housing 18 is made of a rigid, durable and light-weight material such as, but not limited to: metals such as iron, steel, aluminum, titanium, and brass; plastics such as ethylene-vinyl acetate; acrylics such as acrylonitrile-butadiene-styrene and acrylic-styrene-acrylonitrile; polymers such as polycarbonate, polyurethane, polythylene, polybutylene, polyvinyl chloride, polyphenylene oxide, chlorinated polyvinyl chloride, polyamides, and polybutylene terephthalate; carbon fiber; graphite; and any other rigid, durable and light-weight material known to those skilled in the art. The housing 18 may be formed in one of many ways known to those skilled in the art, such as die-casting, machine forming, traditional molding, and blow-molding. The housing 18 acts as a means for storing any electronics located within the glucose meter and acts as a means for mounting items such as the LCD 24, the touch screen 26 and the power button 22. In the exemplary embodiment shown in FIGS. 1 through 3, the housing 18 includes a first, or front, portion 30 and a second, or rear, portion 32 assembled together to house the LCD 24, the touch screen 26 and other components of the glucose meter 10. The front portion 30 includes the window 28 for the LCD 24 and the touchscreen 26.

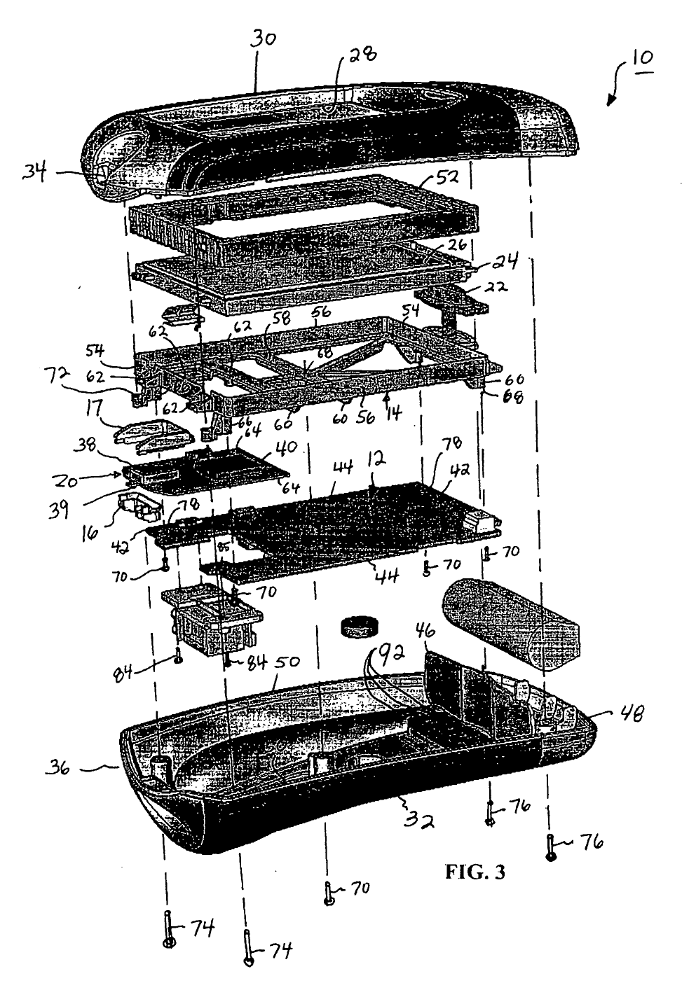

As shown in FIGS. 1 through 3, the glucose meter 10 further includes a port 34 in the housing 18 for receiving a fluid sample. In the exemplary embodiment shown, the port 34 is formed in the front portion 30 of the housing 18 at a top end 36 of the meter 10. The fluid sample (not shown) may comprise, for example, a drop of blood placed on disposable test strip, such as the Ascensia ELITES® Blood Glucose Test Strips. As shown best in FIGS. 2 and 3, the sensor assembly 20 is positioned within the housing 18 and adjacent to the port 34. The sensor assembly 20 includes an electrochemical sensor 38 mounted on an auxiliary printed circuit board (PCB) 40. The electrochemical sensor 38 is adapted to receive a test strip inserted into the port 34 and measure a glucose concentration of a blood sample placed on the test strip. An example of an electrochemical sensor 38 is a sensor that may be used is an amperometric monitoring system. Examples of an electrochemical sensor that can be used to measure glucose concentrations are those used in Bayer Corporation's Ascensia ENTRUS™, CONTOURTM™, DEXS® and ELITES® systems.

As shown in FIGS. 2,3, 6 and 7, the sensor 38 also includes at least one temperature sensing element (e.g., a thermistor, thermometer, or thermocouple device) 39 mounted on a lower surface of the auxiliary PCB 40 and which is used to measure the ambient temperature of the glucose meter 10. As with any chemical sensing method, transient changes in temperature during or between measurement cycles can alter background signal, reaction constants and/or diffusion coefficients. Accordingly, the temperature sensing element 39 is used to monitor changes in temperature over time. A maximum temperature change over time threshold value can be used to invalidate a measurement. Such a threshold value can, of course, be set at any objective level, which in turn can be empirically determined depending upon the particular extraction/sensing device used, how the temperature measurement is obtained, and the analyte being detected. Absolute temperature threshold criteria can also be employed, wherein detection of high and/or low temperature extremes can be used in a data screen to invalidate a measurement. The temperature sensing element 39, for example, may provide a voltage proportional to the temperature to an A/D converter of a microprocessor of the glucose meter, which can then make a determination as to whether the temperature of the testing environment is within predetermined thresholds, and prohibit a user from running a test if accuracy would be negatively affected.

Still referring to FIGS. 2, 3, 6 and 7, the glucose meter 10 is also provided with seals 16, 17 that enclose the port 34 and provide a fluid-tight seal between the port 34 and the internal components of the glucose meter 10 other than the sensor 38 and the temperature sensing element 39. The seals also thermally insulate the sensor 38 and the temperature sensing element 39 from any of the heat-generating internal components of the glucose meter 10 and, in particular, the backlighting of the LCD 24. The seals 16, 17 are made of thermally insulating and electrically insulating elastomeric materials. The seals 16, 17 provide electrical isolation, thermal isolation and a fluid-tight seal and are compressed between the auxiliary PCB 40 and the housing 18 when the glucose meter 10 is assembled.

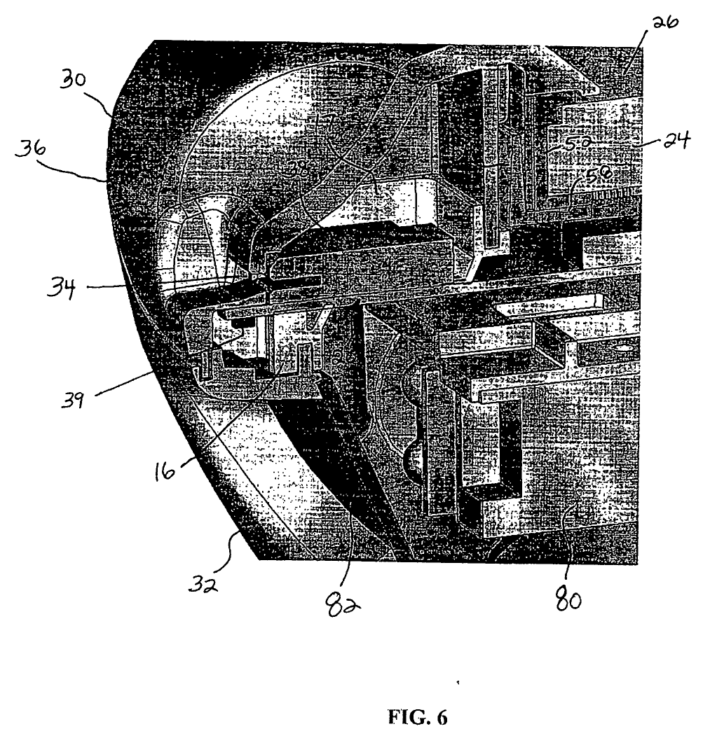

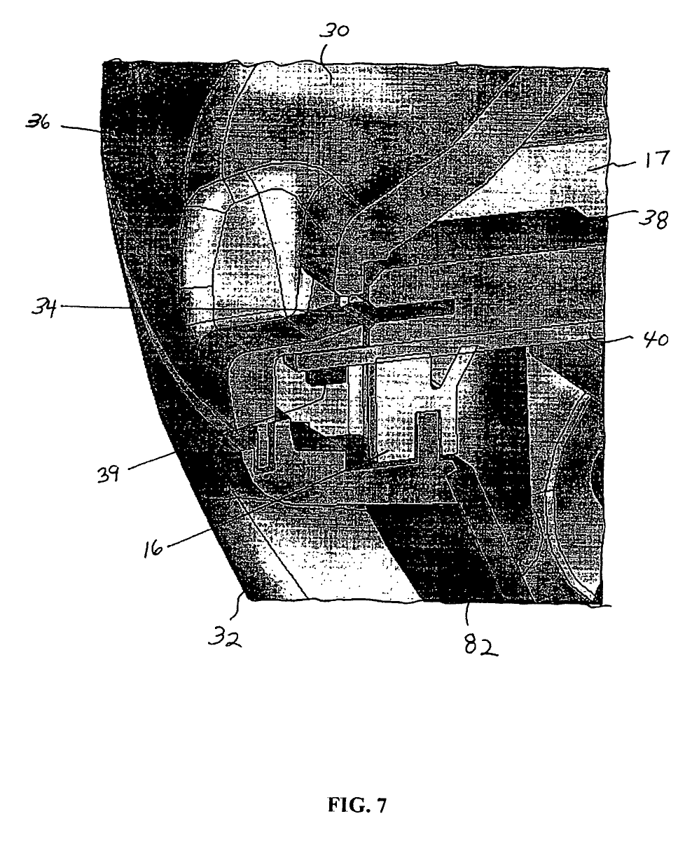

As shown best in FIGS. 6 and 7, the auxiliary PCB 40 of the sensor assembly 20 is positioned in the glucose meter 10 so that a portion of a front surface of the auxiliary PCB 40 of the sensor assembly 20 is pressed against the housing 18 so that the temperature sensing element 39 mounted on the opposite back surface of the auxiliary PCB 40 can more accurately measure the ambient (i.e., outside) temperature of the glucose meter 10. In addition to providing a thermal insulator for the temperature sensing element 39, the seal 16 is compressed between the rear portion 32 of the housing 18 and the auxiliary PCB 40 and acts to press the auxiliary PCB 40 against the front portion 30 of the housing 18 so that the temperature sensing element 39 has direct thermal contact with the outside of the glucose meter 10 and can more accurately measure the ambient temperature of the glucose meter 10. Heat sink grease is also provided between the auxiliary PCB 40 and the housing 18 to reduce any thermal resistance between the auxiliary PCB 40 and the housing 18.

The glucose meter 10 also includes the primary printed circuit board (PCB) 12, which is shown best in FIGS. 2 and 3. Although not viewable, the primary PCB 12 supports much of the electronic components of the glucose meter 10 including a computer processing unit (CPU). The CPU is connected, for example, to the LCD 24, the touch screen 26, the power button 22 and the glucose sensor 38 and is programmed to operate all of the components of the glucose meter 10.

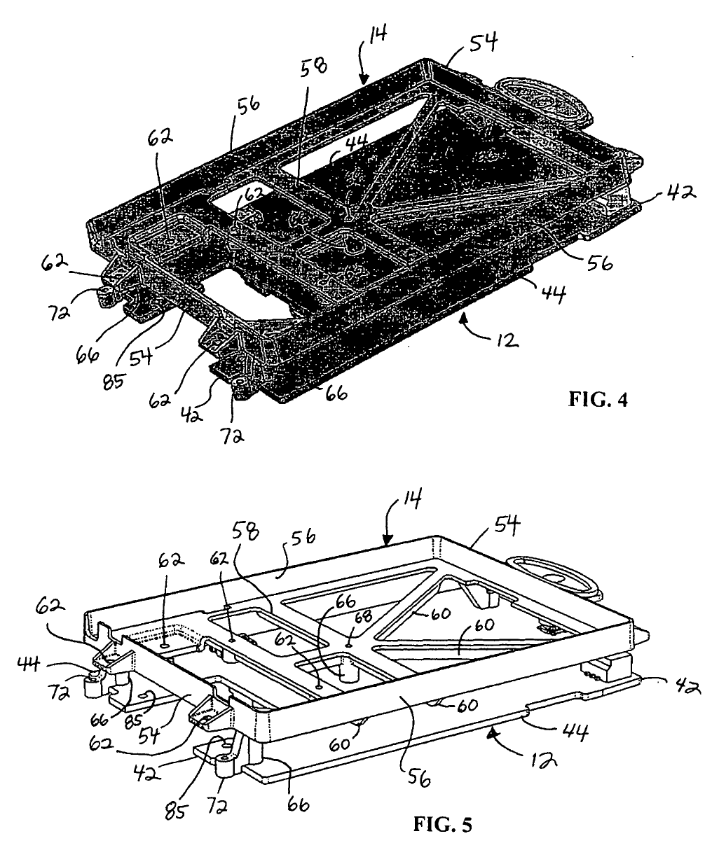

The primary PCB 12, which is also shown in FIGS. 4 and 5, is rigid and flat and generally rectangular, having a length extending between opposing ends 42 and a width extending between opposing sides 44. As shown best in FIG. 2, the opposing ends 42 of the primary PCB 12 extend from the top end 36 of the housing 18 to a bulkhead 46 of the housing 18, which is located near a bottom end 48 of the housing 18. The opposing sides 44 of the primary PCB 12 extend between sides 50 of the housing 18.

In general, a rigid PCB comprises a thin plate on which chips and other electronic components are fixed by solder. A rigid PCB is normally made of continuous woven glass cloth impregnated with epoxy resin, and a layer of metal (usually copper) printed circuit is applied to at least one side of the PCB. A PCB, for example, may comprise a 1/32 inch laminate with 1 ounce copper per square foot. The simplest kind of PCB has components and wires on one side and interconnections (the printed circuit) on the other. The connections are metal strips (usually copper). The pattern of connections is often produced using photo-resist and acid etching. Component leads and integrated circuit pins may pass through holes (“vias”) in the board or they may be surface mounted, in which case no holes are required (though they may still be used to connect different layers). PCBs may also have components mounted on both sides and may have many internal layers, allowing more connections to fit in the same board area. Boards with internal conductor layers usually have “plated-through holes” to improve the electrical connection to the internal layers.

As shown best in FIGS. 2 and 3, the touch screen 26 and the LCD 24 are held together with an elastomeric gasket 52 that runs along a periphery of the touch screen and the LCD and forms a fluid tight seal between the touch screen 26 and the LCD 24 and the window 28 of the front portion 30 of the housing 18. The elastomeric gasket 52 also acts to insulate the touch screen 26 and the LCD 24 from potentially damaging shock and vibration. The touch screen 26 and the LCD 24 are rectangular and, together with the gasket 52, approximate the length and width dimensions of the primary PCB 12.

The glucose meter 10 also includes the internal frame 14, which supports and receives the touch screen 26, the LCD 24 and the gasket 52. The frame 14 is made from a strong and rigid material, such as, but not limited to, metals such as aluminum, plastics such as ethylene-vinyl acetate, acrylics such as acrylonitrile-butadiene-styrene and acrylic-styrene-acrylonitrile, polymers such as polycarbonate, polyurethane, polythylene, polybutylene, polyvinyl chloride, polyphenylene oxide, chlorinated polyvinyl chloride, polyamides, and polybutylene terephthalate, carbon fiber, graphite, and any other suitably strong and rigid material known to those skilled in the art.

The frame 14, which is also shown in FIGS. 4 and 5, is rectangular and includes opposing end walls 54 and opposing side walls 56 and approximates the length and width dimensions of the primary PCB 12. The frame 14 also includes a base wall 58 extending between the end walls 54 and the side walls 56. Much of the base wall 58 is removed (or simply not formed) during the fabrication of the frame 14 in order to reduce the weight of the frame 14 without significantly reducing the strength or the torsional rigidity of the frame 14. Ribs 60 are located on a lower surface of the base wall 58 to provide additional strength and rigidity.

As shown best in FIGS. 3 through 5, the frame 14 includes holes 62 for receiving screws or other suitable fasteners (not shown) passing through holes 64 in the auxiliary PCB 40 of the sensor assembly 20 and securing the auxiliary PCB to the frame 14. The frame 14 also includes bosses 66 extending from the lower surface of the base wall 58 and holes 68 extending through the base wall 58 and the bosses 66 for receiving screws 70 or other suitable fasteners securing the primary PCB 12 to the frame 14. Some of the same screws 70 that secure the primary PCB 12 and the frame 14 together also pass through the housing 18 and secure the front and the rear portions 30, 32 of the housing 18 together upon assembly of the glucose meter 10. The touch screen 26, the LCD 24 and the gasket 52 are secured between the frame 14 and the front portion 30 of the housing 18 upon assembly of the glucose meter 10. The frame 14 further includes holes 72 for receiving screws 74 securing the housing 18 together and to the frame 14. Additional screws 76 secure the bottom end 48 of the housing 18 together.

As shown best in FIG. 3, the primary PCB 12 includes holes 78 which receive the screws 70 securing the primary PCB 12 to the frame 14 and the housing 18. Connecting the primary PCB 12 to the frame 14 increases the stiffness and the torsional rigidity of the glucose meter 10 and, therefore, helps to protect the touch screen 26 and the LCD 24 upon the glucose meter 10 being accidentally dropped. Securing the primary PCB 12 to the frame 14 has been found to substantially improve the ruggedization of the glucose meter 10 and allow the glucose meter 10 to survive an accidental drop from a height of about five feet onto a hard surface without damage and continue to function properly.

As shown best in FIGS. 2 and 3, the exemplary embodiment of the glucose meter 10 includes a barcode scanner 80 for scanning barcodes off disposable test strips used with the glucose meter 10. As also shown in FIG. 1, the housing 18 includes a window 82 for the barcode scanner 80. In the exemplary embodiment shown, the barcode scanner 80 is secured to the primary PCB 12 with suitable fasteners, such as screws 84. The PCB is provided with holes 85 for receiving the barcode scanner 80.

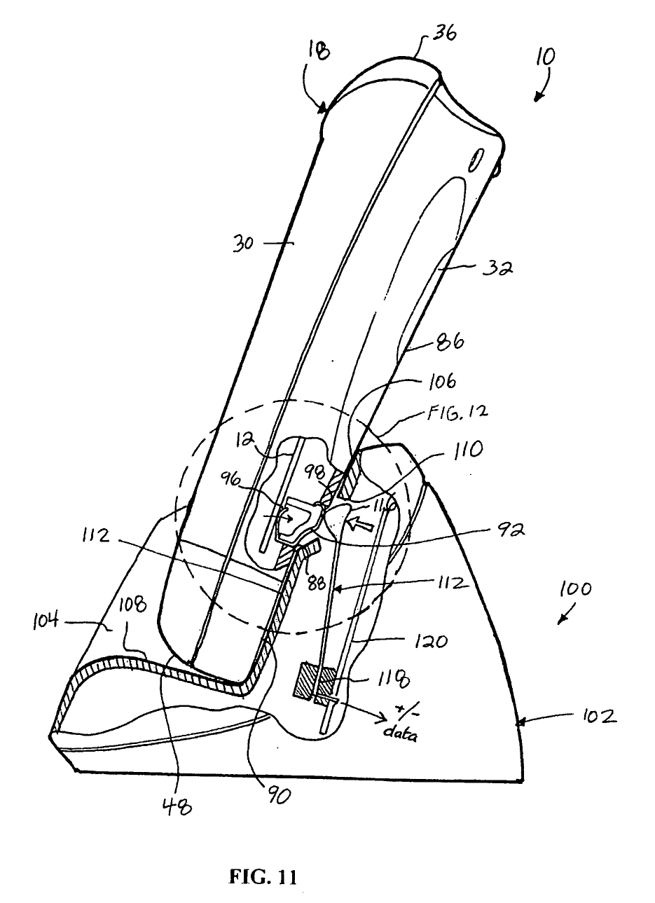

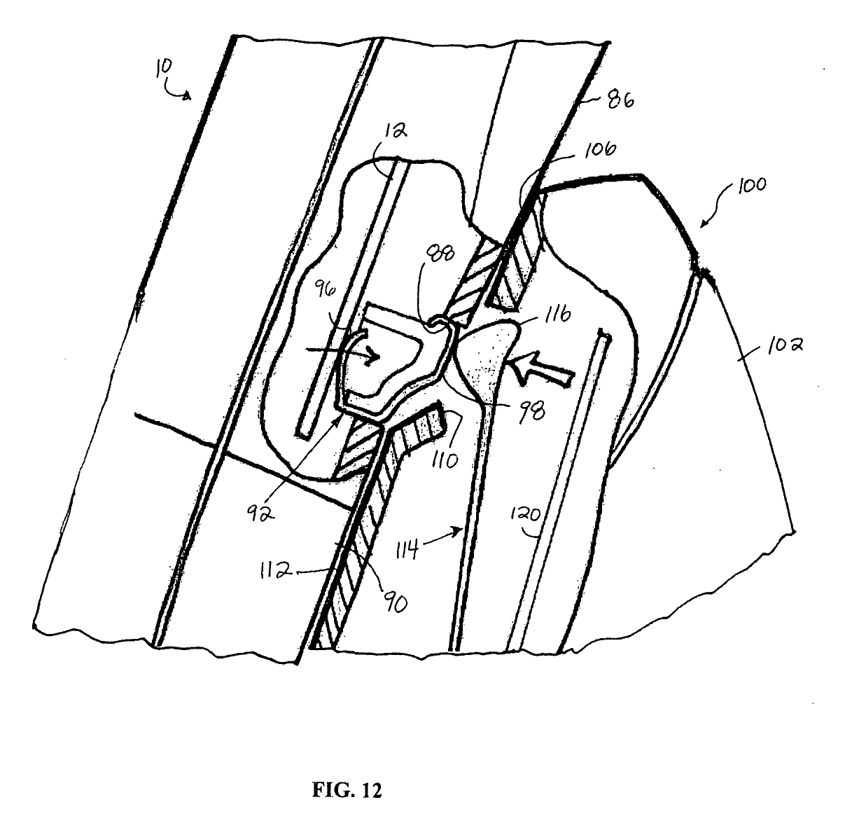

FIGS. 8 through 10 show the glucose meter 10 of FIG. 1 being received in the docking station 100, and FIGS. 11 and 12 are side elevation views, partially cut-away, of the handheld glucose meter 10 received in the docking station 100. The glucose meter 10 and the docking station 100 of the present disclosure are both ruggedized and durable. In addition, the glucose meter 10 and the docking station 100 include novel features that allow the glucose meter 10 to be easily received in the docking station 100, yet reduce wear create by the docking process such that the docking station 100 and the glucose meter 10 can withstand thousands of docking cycles (e.g., 9,000 to 18,000 cycles) and continue to function properly. The glucose meter 10 and the docking station 100 together comprise a system.

As shown best in FIG. 2, the housing 18 of the glucose meter 10 includes a wall 86 extending upwardly from the bottom end 48 of the housing 18, and an opening 88 in the wall 86 spaced from the bottom end 48. The wall 86 defines a concave depression 90 extending between the bottom end 48 and the opening 88. The glucose meter 10 also includes at least one electrically conductive contact 92 in contact with the primary PCB 12 and extending through the opening 18 of the housing 18. The contact 92 of the glucose meter 10 provides a data connection to the CPU of the glucose meter 10 and also provides a electrical connection to a rechargeable battery 94 (shown in FIGS. 2 and 3) of the glucose meter 10.

The docking station 100 includes an external housing 102 defining a pocket 104 for receiving the medical diagnostic apparatus 10. The pocket 104 includes a wall 106 extending upwardly from a bottom end 108 of the pocket 104 for slidingly receiving the wall 86 of the glucose meter 10 when the bottom end 48 of the meter 10 is received in the pocket 104. As shown best in FIG. 8, the wall 106 of the pocket 104 includes at least one opening 110 spaced from the bottom end 108 of the pocket 104 and a convex projection 112 extending from the bottom end 108 of the pocket 104 to the opening 110 in the wall 106 of the pocket 104. The convex projection 112 of the docking station 100 is sized and shaped to mate with the concave depression 90 of the glucose meter 10 when the bottom end 48 of the meter 10 is received in the pocket 104 of the docking station 100, as shown best in FIGS. 11 and 12. Still referring to FIGS. 11 and 12, the docking station 100 also includes at least one electrically conductive contact 114 mounted within the housing 102 of the docking station 100 and extending through the opening 110 of the housing of the docking station 100 so that, when the glucose meter 10 is received in the pocket 104, the contact 114 extending out of the docking station 100 touches the contact 92 extending out of the glucose meter 10 to provide an electrical connection between the docking station 100 and the glucose meter 10.

The convex projection 112 of the docking station 100 and the concave depression 90 of the glucose meter 10 have at least two functions. First, the projection 112 and the depression 90 mate and ensure that the glucose meter 10 is correctly positioned in the docking station 100 when the meter is deposited into the pocket 104 of the docking station 100, such that the contact 114 of the docking station 100 is in contact with the contact 92 of the glucose meter 10. In addition, the concave depression 90 of the glucose meter 10 prevents the contact 114 of the docking station 100 from rubbing against the wall 86 of the housing 18 of the glucose meter 10 when the bottom end 48 of the glucose meter 10 is deposited into the pocket 104 of the docking station 100, thereby preventing unnecessary wear and damage to the contact 114 of the docking station 100 and the housing 18 of the glucose meter 10.

The glucose meter 10 includes a plurality of the contacts 92 extending through the opening 88 of the meter, and the docking station 100 includes a plurality of the openings 110 and a plurality of the contacts 114 extending through the openings of the docking station 100. In the exemplary embodiment show, the docking station 100 and the glucose meter 10 each include twelve contacts 92, 114. The contacts 92 of the glucose meter 10 are fixed in place and substantially immovable. As shown best in FIGS. 11 and 12, the contacts 92 of the glucose meter 10 each comprise a metal strip bent into a U-shape and having a first free end 96 in contact with the primary PCB 12 of the meter 10 and a second fixed end 98 for contact with the contacts 114 of the docking station 100. The primary PCB 12 of the glucose meter 10 is not attached to the contacts 92 of the glucose meter 10, but instead has leads that are in electrical contact with the free ends 96 of the contacts 92 of the glucose meter 10 so that, if the glucose meter 10 is dropped for example, the PCB 12 can move without breaking a connection between the PCB 12 and the contacts 92. In addition, the second fixed ends 98 of the contacts 92 can easily be cleaned, yet prevent fluid ingress into the housing 18 of the glucose meter 10.

The contacts 114 of the docking station 100 include a free end 116 that is movable and biased out of the docking station 100. The contacts 114 of the docking station 100 each comprise an elongated metal strip having a fixed end 118 secured to a PCB 120 of the docking station 100 and the free end 116 extending out of the opening 110 of the housing 102 of the docking station 100. The free end 116 of each of the metal strips 114 is twisted so that a thin edge of the strip faces out of the docking station 100 through its respective opening 110. According to one exemplary embodiment, the elongate strips 114 of the docking station 100 are each about 30 millimeters long and each provide a spring force of about 0.15 N to about 0.4 N against the strips 92 of the glucose meter 10.

Faces of the second fixed ends 98 of the metal strip contacts 92 of the glucose meter 10 face out of the glucose meter 10 and contact the thin edges of the free ends 116 of the contacts 114 of the docking station 100 when the glucose meter 10 is received in the docking station 100. Because the thinner edges of the contacts 114 of the docking station 100 contact the wider faces of the contacts 92 of the glucose meter 10, there is a large positional tolerance between the contacts 92, 114 and the contacts will remain in contact and provide a reliable electrical connection even if the glucose meter 10 fits slightly loosely in the docking station 100 during docking. In one exemplary embodiment, the thin edge of the contacts 114 of the docking station 100 each have a width of about 0.4 mm and the faces of the contacts 92 of the glucose meter 10 each have a width of about 2 mm, so that there is a 0.75 mm tolerance between the contacts 92, 114.

The present disclosure, therefore, provides a new and improved portable medical diagnostic apparatus that can withstand being accidentally dropped by a user and continue to function properly, and has good heat insulating properties to ensure accurate operation. The present disclosure also provides a new and improved docketing station that is itself ruggedized and provides an easy and reliable electrical docking connection with the portable medical diagnostic apparatus.

Numerous further modifications and alternative embodiments of the disclosure will be apparent to those skilled in the art in view of the foregoing description. This description is to be construed as illustrative only, and is for the purpose of teaching those skilled in the art the best mode of carrying out the disclosure. The details of the apparatus and method may be varied substantially without departing from the spirit of the disclosure, and the exclusive use of all modifications which come within the scope of the appended claims is reserved.

What is claimed is:

1. A medical diagnostic apparatus comprising:
a housing;
a sensor assembly located within the housing and including a temperature sensing element mounted on a printed circuit board (PCB); and
at least one thermal seal compressed between the PCB of the sensor assembly and the housing and separating the temperature sensing element from any heat-generating components contained in the housing.

2. An apparatus according to claim 1, wherein the thermal seal is made of thermally insulating elastomeric material.

3. An apparatus according to claim 2, wherein the thermal seal is made of electrically insulating material.

4. An apparatus according to claim 3, wherein the thermal seal provides a fluid-tight seal between the temperature sensing element and the heat-generating components contained in the housing.

5. An apparatus according to claim 1, wherein the housing includes a port adjacent the sensor assembly and the thermal seal is arranged and adapted to provide a fluid-tight seal between other components contained in the housing and the port and -the sensor assembly.

6. An apparatus according to claim 1, wherein the housing comprises a first portion and a second portion secured together and the PCB is secured between the first and the second portions of the housing, and wherein the thermal seal is compressed between the second portion of the housing and the PCB and biases the PCB against the first portion of the housing.

7. An apparatus according to claim 6, further comprising heat sink grease provided between the PCB and the housing.

8. An apparatus according to claim 6, wherein the temperature sensing element is positioned on the PCB between the second portion of the housing and the PCB, and between the seal and the housing.

9. An apparatus according to claim 1, wherein the housing includes a port adjacent the sensor for allowing a test strip to be inserted into the housing and into the sensor, and the thermal seal surrounds the sensor and the port.

10. An apparatus according to claim 1, the PCB is in direct contact with the housing and heat sink grease is provided between the PCB and the housing.

11. An apparatus according to claim 1, wherein the sensor assembly comprises a glucose sensor.

12. An apparatus according to claim 1, further comprising:
a primary printed circuit board (PCB) positioned within the housing, wherein the PCB is rigid, flat and generally rectangular and has a length extending between opposing ends and a width extending between opposing sides, and wherein the PCB of the sensor assembly comprises an auxiliary PCB;
a liquid crystal display (LCD) positioned within the housing adjacent a window of the housing, wherein the LCD includes a length and a width that approximates the length and the width of the primary PCB; and
a rigid frame supporting the LCD and having a length and a width that approximates the length and the width of the primary PCB, wherein the rigid frame is secured to the primary PCB and at least one of the frame and the primary PCB are secured to the housing.

13. A medical diagnostic system including an apparatus according to claim 1, and further comprising:
a docking station having a housing defining a pocket sized and shaped for receiving the medical diagnostic apparatus, and wherein the pocket includes a bottom end and at least one opening spaced from the bottom end of the pocket and a convex projection extending from the bottom end of the pocket to the opening in the pocket, and the docking station also includes at least one electrically conductive contact extending through the opening of the pocket; and
wherein the housing of medical diagnostic apparatus further includes a bottom end, at least one opening spaced from the bottom end of the medical diagnostic apparatus, and a concave depression extending between the bottom end and the opening, and wherein the concave depression of the apparatus is sized and shaped to mate with the convex projection of the docking station when the apparatus is received in the pocket, and the medical diagnostic apparatus also includes at least one electrically conductive contact extending through the opening of the housing of the apparatus so that, when the apparatus is received in the pocket of the docking station, the contact extending out of the docking station contacts the contact extending out of the apparatus.

14. A medical diagnostic apparatus comprising:
a housing;
a primary printed circuit board (PCB) positioned within the housing, wherein the PCB is rigid, flat and has a length extending between opposing ends and a width extending between opposing sides;
a liquid crystal display (LCD) positioned within the housing adjacent a window of the housing, wherein the LCD includes a length and a width that approximates the length and the width of the primary PCB; and
a rigid frame supporting the LCD and having a length and a width that approximates the length and the width of the primary PCB, wherein the rigid frame is secured to the primary PCB and at least one of the frame and the primary PCB are secured to the housing.

15. An apparatus according to claim 14, wherein the primary PCB is substantially rectangular.

16. An apparatus according to claim 14, wherein the length and the width of the primary PCB approximates an internal length and width of the housing.

17. An apparatus according to claim 14, wherein the housing comprises a first portion and a second portion secured together and the primary PCB, the frame and the LCD are secured between the first and the second portions of the housing.

18. An apparatus according to claim 14, wherein the primary PCB is secured to the frame and the housing with screws.

19. An apparatus according to claim 14, wherein the frame is rectangular and includes opposing end walls and opposing side walls, a base wall extending between the end walls and the side walls, and ribs located on the base wall.

20. A medical diagnostic system including an apparatus according to claim 14, and further comprising:
a docking station having a housing defining a pocket sized and shaped for receiving the medical diagnostic apparatus, and wherein the pocket includes a bottom end and at least one opening spaced from the bottom end of the pocket and a convex projection extending from the bottom end of the pocket to the opening in the wall of the pocket, and the docking station also includes at least one electrically conductive contact extending through the opening of the pocket; and
wherein the housing of medical diagnostic apparatus further includes a bottom end, at least one opening spaced from the bottom end of the medical diagnostic apparatus, and a concave depression extending between the bottom end and the opening, and wherein the concave depression of the apparatus is sized and shaped to mate with the convex projection of the docking station when the apparatus is received in the pocket, and the medical diagnostic apparatus also includes at least one electrically conductive contact extending through the opening of the housing of the apparatus so that, when the apparatus is received in the pocket of the docking station, the contact extending out of the docking station contacts the contact extending out of the apparatus.

21. An apparatus according to claim 14, further comprising a sensor assembly positioned within the housing and wherein the housing includes a port adjacent the sensor assembly for allowing a test strip to be inserted into the housing and into a sensor of the sensor assembly.

22. An apparatus according to claim 21, wherein the sensor comprises a glucose sensor.

23. An apparatus according to claim 21, wherein the sensor assembly is secured directly to the frame.

24. An apparatus according to claim 21, further comprising a thermal seal compressed between the sensor assembly and the housing.

25. A medical diagnostic system comprising:
a medical diagnostic apparatus having, an external housing having a bottom end, a wall extending upwardly from the bottom end, and at least one opening in the wall spaced from the bottom end, and wherein the wall defines a concave depression extending between the bottom end and the opening,
a board located within the housing, and
at least one electrically conductive contact in contact with the board and extending through the opening of the housing; and
a docking station having,
an external housing defining a pocket sized and shaped for receiving the medical diagnostic apparatus, and wherein the pocket includes a wall extending upwardly from a bottom end of the pocket for slidingly receiving the wall of the medical diagnostic apparatus when the apparatus is received in the pocket, and wherein the wall of the pocket includes at least one opening spaced from the bottom end of the pocket and a convex projection extending from the bottom end of the pocket to the opening in the wall of the pocket, and wherein the convex projection of the docking station is sized and shaped to mate with the concave depression of the apparatus when the apparatus is received in the pocket, and
at least one electrically conductive contact mounted within the housing of the docking station and extending through the opening of the housing of the docking station so that, when the apparatus is received in the pocket, the contact extending out of the docking station contacts the contact extending out of the apparatus.

26. A system according to claim 25, wherein the medical diagnostic apparatus includes a plurality of the contacts extending through the opening in the wall of the apparatus, and the docking station includes a plurality of the openings and a plurality of the contacts extending through the openings in the wall of the docking station.

27. A system according to claim 25, wherein the contact of the medical diagnostic apparatus is substantially immovable.

28. A system according to claim 25, wherein the contact of the docking station is movable and biased out of the docking station.

29. A system according to claim 25, wherein the board within the medical diagnostic apparatus comprises a printed circuit board electrically connected to the contacts of the medical diagnostic apparatus, and the docking station includes a printed circuit board contained within the housing of the docking station and electrically connected to the contacts of the docking station.

30. A system according to claim 29, wherein the medical diagnostic apparatus includes a rechargeable battery electrically connected to the contacts of the medical diagnostic apparatus.

31. A system according to claim 25, wherein the medical diagnostic apparatus comprises a glucose meter.

32. A system according to claim 25, wherein the contact of the docking station comprises an elongated metal strip having a fixed end secured to the board of the docking station and a free end extending out of the opening in the housing of the docking station and wherein the free end of the metal strip is twisted so that a thin edge of the strip faces out of the docking station.

33. A system according to claim 32, wherein the contact of the medical diagnostic apparatus comprises a metal strip and wherein a face of the metal strip faces out of the apparatus and contacts the thin edge of the strip of the docking station when the medical diagnostic apparatus is received in the docking station.

34. A system according to claim 33, wherein the thin edge of the strip of the docking station has a width of about 0.4 mm and the face of the metal strip of the medical diagnostic apparatus has a width of about 2 mm.

35. A system according to claim 33, wherein the strip of the docking station provides a spring force of about 0.15 N to about 0.4 N against the strip of the medical diagnostic apparatus.
